(19) United States
(12) Patent Application Publication
(10) Pub. No.: US 2003/0187267 A1
(43) Pub. Date: Oct. 2, 2003

(54) Novel 1,2,3,4-tetrahydroisoquinoline, their preparation method and their use as fungicides

(76) Inventors: Didier Babin, Montigny (FR); Yannick Benedetti, Rosny Sous Bois (FR); Fabienne Chatreaux, Neuilly Sur Marne (FR); John Bernard Weston, Maisons Laffitte (FR)

Correspondence Address: MUSERLIAN AND LUCAS AND MERCANTI, LLP, 600 THIRD AVENUE, NEW YORK, NY, 10016, US

(21) Appl. No.: 10/240,014

(22) PCT Filed: Apr. 4, 2001
(86) PCT No.: PCT/FR01/01004

(30) Foreign Application Priority Data
Apr. 5, 2000 (FR) 00/04324

(51) Int. Cl. C 07D 4 7/14

(52) U.S. Cl. 546/148

(57) ABSTRACT

The invention concerns compounds of formula (I) wherein: p=1 or 2; X, X₁ and X₂ represent N or CH═; R₁, R₂, R₃, R₄, R₅ and R₆ represent a hydrogen atom, a halogen atom, alkyl, O-alkyl, S—(O)ₙalkyl, alkenyl, O-alkenyl, S—(O)ₙalkenyl, alkynyl, O-alkynyl, S—(O)ₙalkynyl; n=0, 1 or 2, or NO₂, NH₂ or C═N or R₁, R₂, R₃ or R₅, R₆ form a cycle, or R₄A can be cycloalkyl, heterocycle, aryl, O-aryl or oxygenated or nitrogenated chain; R₇ represents H, OH, SO₃H or OPO(OH)₂; A and B=hydrogen or oxygenated or nitrogenated chain; C and D=hydrogen, halogen or alkyl or together form with the carbons bearing them a cycle. The compounds of formula (I) have antifungal properties.

The present invention relates to new derivatives of 1,2,3,4-tetrahydroisoquinoline, their preparation process and their use as fungicides.

A subject of the invention is, in all possible stereoisomeric forms, as well as their mixtures, the compounds of formula (I):

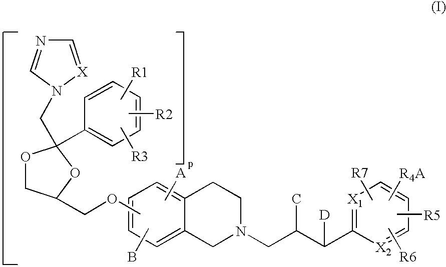

in which

p represents the number 1 or 2,

X, X₁ and X₂, identical or different, represent a nitrogen atom or a CH═ radical,

R₁, R₂, R₃, R₅ and R₆, identical to or different from each other, in any position on the rings which carry them, represent a hydrogen atom, a halogen atom, an alkyl, O-alkyl, S(O)ₙ-alkyl, alkenyl, O-alkenyl, S(O)ₙ-alkenyl, alkynyl, O-alkynyl, S(O)ₙ-alkynyl radical containing up to 8 carbon atoms, optionally substituted by one or more halogen atoms, n representing the number 0, 1 or 2, or represent an NO₂, NH₂ or C≡N radical, R₁, R₂ and R₃ on the one hand and R₅ and R₆ on the other hand being able to form rings two by two,

R₇ represents a hydrogen atom or an OH, OSO₃H or OPO(OH)₂ radical,

R₄A represents an R₄ radical which can take one of the values indicated above for R₁, R₂, R₃, R₅ or R₆, and also being able to represent a non substituted or substituted heterocycle, a non substituted or substituted aryl or O-aryl group containing up to 14 carbon atoms, a cycloalkyl containing 3 to 6 carbon atoms, non substituted or substituted by an aryl optionally substituted by one or more halogen atoms and also being able to represent an oxygenated or nitrogenous chain connected to the phenyl or heteroaryl nucleus by an oxygen or nitrogen atom,

A and B, identical to or different from each other, represent a hydrogen atom or an oxygenated or nitrogenous chain connected to the phenyl nucleus by an oxygen or nitrogen atom,

C and D, identical to or different from each other, represent a hydrogen or halogen atom or an alkyl radical containing up to 8 carbon atoms, optionally substituted by one or more halogen atoms or, together with the carbons which carry them, form a ring optionally substituted by one or more halogen atoms, one or more alkyl radicals containing up to 8 carbon atoms or together form a double bond, with the exception of the compounds corresponding to formula (IA):

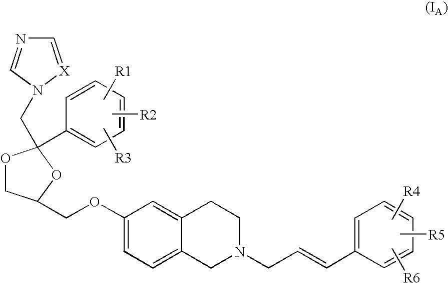

in which the various substituents retain their previous meaning, as well as their addition salts with acids. The compounds of formula (I_{A}) are described and claimed in European Patent Application No. 992502 filed on Oct. 5, 1999 by the Applicant company.

Of course, when X₁ and X₂ both represent a nitrogen atom, at least one of the substituents R₄A, R₅, R₆ or R₇ of the heteroaryl radical represents a hydrogen atom.

Among the addition salts with acids, there can be mentioned those formed with mineral acids, such as hydrochloric, hydrobromic, sulphuric or phosphoric acids or with organic acids such as formic, acetic, trifluoroacetic, propionic, benzoic, citric, maleic, fumaric, succinic, tartaric acid, alkane-sulphonic acids, such as methane or ethane sulphonic acid, arylsulphonic acids such as benzene or paratoluenesulphonic acid.

Substituents A and B are preferably in position 5 or 7.

In the definition of the substituents, the alkyl, alkenyl or alkynyl radical is preferably a methyl, ethyl, propyl, isopropyl, n-butyl, isobutyl, terbutyl, decyl or dodecyl, vinyl, allyl, ethynyl, propynyl radical, or a cyclic radical such as cyclopropyl, cyclobutyl, cyclopentyl or cyclohexyl,

the halogen is preferably fluorine or chlorine, or bromine,

when C and D form a cyclic radical, it can for example be a cyclopropyl radical optionally substituted by a fluorine atom or by a gem-dimethyl,

the aryl radical is preferably the phenyl radical,

the heterocycle radical can contain one or more heteroatoms, it is preferably a radical with 5 or 6 members optionally containing one or two double bonds, and one or more nitrogen atoms, such as for example

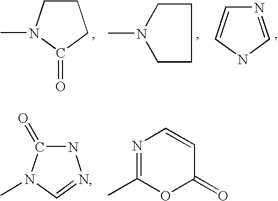

each of these radicals being able to be substituted, it can in particular be the

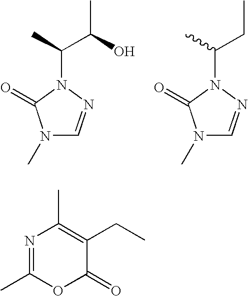

radicals.

Among the preferred compounds of the invention, there can be mentioned the compounds of formula (I₁):

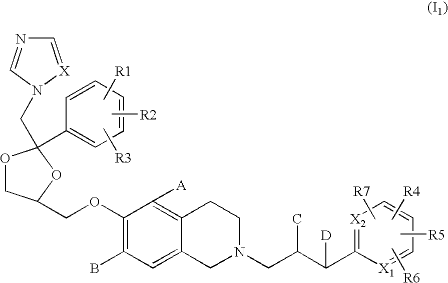

in which X, X₁ and X₂, identical or different, represent a nitrogen atom or a —CH═ radical, R₁, R₂, R₃, R₅, R₆, R₇, identical or different, being in any position on the rings which carry them, those in which X, X₁ and X₂ represent a —CH═ radical, those in which X represents a CH═ radical and either X₁ or X₂ represents a nitrogen atom, those in which R₁ and R₂ represent a halogen atom, those in which R₄ is a halogen atom, and more particularly those in which R₁ and R₂ and/or R₄ represent a chlorine atom, those in which R₃ represents a hydrogen atom, those in which C and D form a double bond, and among these, particularly, those in which the geometry of the double bond is E, those in which the dioxoxaryl radical is in cis position.

A more particular subject of the invention is the compounds of formula (I) defined above in which R₇ represents an OH, OSO₃H or OPO(OH)₂ radical.

A quite particular subject of the invention is the compounds of formula (I), in which A or B does not represent a hydrogen atom.

Among the preferred compounds of the invention, there can quite particularly be mentioned those in which A or B represents a

W(CO)ᵣ(CH₂)ₛZ

radical in which r represents the number 0 or 1, s represents an integer varying from 0 to 6, W represents an oxygen atom or an —N(R₁₁)— radical, R₁₁ representing a hydrogen atom or an alkyl radical containing up to 8 carbon atoms, R₁₁ also being able to form a ring together with the nitrogen atom which carries it and another atom of the (CO)ᵣ(CH₂)ₛ—Z chain, Z represents a hydrogen atom, an SO₃H or OSO₃H, PO(OH)₂ or OPO(OH)₂, or CO₂H radical as well as the alkaline, alkaline earth salts or salts of amines of these radicals, or a

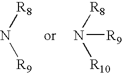

radical,

R₈ and R₉ representing a hydrogen atom, an alkyl radical containing up to 8 carbon atoms, R₁₀ representing an alkyl radical containing up to 8 carbon atoms, R₈ and R₉ being able to form a ring optionally containing another heteroatom, and optionally being substituted, or Z represents an optionally substituted heterocyclic radical.

Thus A and B can in particular represent

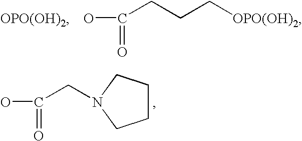

OSO₃H, O-sugar (pyranose, furanose), a

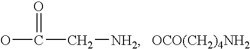

radical.

A quite particular subject of the invention is the compounds of formula (I), in which W represents a nitrogen atom.

In this case, A and B can represent an NHCOOR′₁, NHCOR′₂, NH—CONR′₃R′₄ chain,

R′₁, R′₂, R′₃ and R′₄ representing alkyl radicals containing up to 8 carbon atoms

NHCOCO₂H, NH—CO₂P(O)(OH)₂, NHSO₃H, NHPO₃H₂,

as well as the alkaline, alkaline earth salts and salts of amines of the latter two compounds

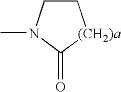

with a=1 or 2.

When R₄A represents an oxygenated or nitrogenous chain, it is preferably one of the preferred values indicated above for A and B, or also one of the heterocycles with 5 or 6 members containing one or more nitrogen atoms and one or more double bonds.

A more particular subject of the invention is the compounds of Examples 3, 6, 10, 12 and 13.

The compounds of formula (I) have useful antifungal properties; they are in particular active against Candida albicans and other Candida such as Candida glabrata, krusei, tropicalis, pseudotropicalis and parapsilosis, on Aspergillus fumigatus, Aspergillus flavus, Aspergillus niger, Cryptococcus neoformans, Microsporum canis, Trichophyton rubrun, Trichophyton mentagrophyte.

The compounds of formula (I) can be used as medicaments in man or animals, to combat in particular digestive, urinary, vaginal or cutaneous candidosis, cryptococcosis, for example neuromeningeal, pulmonary or cutaneous cryptococcosis, bronchopulmonary and pulmonary aspergillosis and invasive aspergillosis of the immunosuppressed.

The compounds of the invention can also be used in the prevention of mycotic illnesses in the congenital or acquired immunosuppressed.

The compounds of the invention are not limited to pharmaceutical use, they can also be used as fungicides in other fields than the pharmaceutical field.

A subject of the invention is therefore as antifungal compounds, the compounds of formula (I).

A subject of the invention is also the compounds of formula (I) as well as their pharmaceutically acceptable salts, as medicaments.

A quite particular subject of the invention is pharmaceutical compositions containing as active ingredient at least one compound of formula (I) or one of its pharmaceutically acceptable salts.

These compositions can be administered by buccal, rectal, parenteral route or by local route as a topical application on the skin and the mucous membranes, but the preferred route is the buccal route.

These can be solid or liquid and can be presented in the pharmaceutical forms commonly used in human medicine, such as for example plain or sugar coated tablets, gelatin capsules, granules, suppositories, injectable preparations, ointments, creams, gels; they are prepared according to the usual methods. The active ingredient(s) can be incorporated into the excipients usually used in these pharmaceutical compositions, such as talc, gum arabic, lactose, starch, magnesium stearate, cocoa butter, cyclodextrins, aqueous or non-aqueous vehicles, fatty matter of animal or vegetable origin, paraffin derivatives, glycols, various wetting, dispersing or emulsifying agents, preservatives.

These compositions can also be presented in the form of a powder intended to be dissolved extemporaneously in an appropriate vehicle, for example apyrogenic sterile water.

The dose administered is variable according to the disorder treated, the patient in question, the administration route and the product considered. It can be, for example, comprised between 50 mg and 300 mg per day by oral route in an adult for the product of Example 3 or Example 6.

A subject of the invention is also a process characterized in that a compound of formula (II):

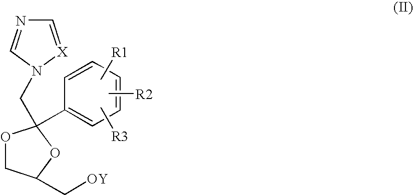

in which:

Y represents a mesyl or tosyl radical

and the other substituents retain their previous meaning is subjected to the action of a compound of formula (III)

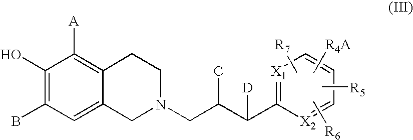

in which the different substituents retain their previous meaning, in order to obtain the corresponding compound of formula (I), which is subjected if desired to the action of a reducing, substitution, addition agent or to the action of an acid in order to obtain the desired compound.

The products of formula (II) used as starting products are generally known products which can be prepared according to the process indicated in J. Med. Chem. 1979 22(8)1003.

Certain products of formula (III) are new products; they can be prepared as indicated in the experimental part.

A subject of the invention is also a process characterized in that a compound of formula (IV):

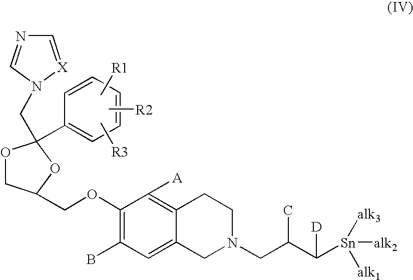

in which alk₁, alk₂ and alk₃ represent an alkyl radical containing up to 8 carbon atoms, R1, R2, R3 and X retain their previous meaning, is subjected to the action of a compound of formula (V):

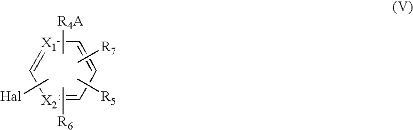

in which Hal represents a halogen atom and the other substituents retain their previous meaning in order to obtain the corresponding compound of formula (I), which is subjected if desired to the action of a reducing, substitution, addition agent or to the action of an acid in order to obtain the desired compound.

The products of formula (IV) are new products which can be prepared as indicated hereafter in the experimental part.

A further subject of the invention is a variant of the previous processes, characterized in that a compound of formula (VI):

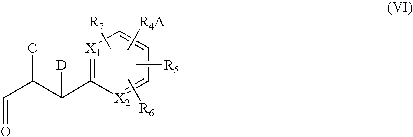

in which the substituents retain the same value as previously, is subjected to the action of a compound of formula (VII):

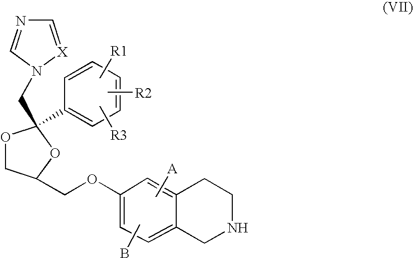

in which the substituents retain the same value as previously, then to the action of a reducing agent in order to obtain the corresponding compound of formula (I) which is subjected if desired to the action of a reducing, substitution, addition agent or to the action of an acid in order to obtain the desired compound.

The compounds of formula (VI) are new products prepared as indicated hereafter in the experimental part.

The following examples illustrate the invention without however limiting it.

Preparation 1: 3-[4-[2,4-dihydro-2-(1-methylpropyl)-3-oxo-3H-1,2,4-triazol-4-yl]phenyl]-2(E)-propenal.

Stage A: 1,1-dimethylethyl 2-[[[(4-iodophenyl)-amino]-carbonyl]-hydrazine-carboxylate.

A mixture of 20 g of 1-iodo-4-isocyanoto-benzene and 100 ml of tetrahydrofuran (THF) is cooled down to 0° C., then 11.84 g of tert-butylcarbazate (BOCNHNH₂) in 100 ml of THF is introduced at a temperature lower than −10° C. Agitation is carried out for 1 hour at 0° C. followed by evaporating under reduced pressure. The reaction medium is taken up in ether and agitated for 2 hours at ambient temperature followed by separating, rinsing and drying at 55° C. 30 g of sought product is obtained, melting at 55° C.

Stage B: N-(4-iodophenyl)-hydrazinecarboxamide.

A mixture containing 30 g of the product of the previous stage, 250 ml of THF and 30 ml of a 6N hydrochloric acid solution is agitated under reflux for 2 hours 30 minutes, followed by cooling down to 0° C. and adding 150 ml of ethyl ether. After agitating for 1 hour 30 minutes at 0° C., the reaction medium is separated, rinsed and dried. 22.69 g of sought product is obtained.

Stage C: 2,4-dihydro-4-(4-iodophenyl)-3H-1,2,4-triazol-3-one.

15.46 g of potash in powder form is added to a mixture of 22.70 g of the product of the previous stage and 247 ml of butanol. After agitating for 30 minutes and adding 16.4 g of formamidine acetate, the reaction medium is taken to 110° C. and maintained at this temperature for 5 hours, then taken to 0° C. 350 ml of water is added followed by agitating for 30 minutes at 0° C., separating, rinsing with water and with ether. After drying at 50° C. under reduced pressure, 13.73 g of product is obtained which melts at 258° C.

Stage D: 2,4-dihydro-4-(4-iodophenyl)-2-(1-methylpropyl)-3H-1,2,4-triazol-3-one.

A mixture of 1.15 g of the product of the previous stage, 10 ml of methylisobutylketone, 0.87 ml of bromobutane, 0.14 g of aliquot 336 and 1.1 g of K₂CO₃ is heated at 110° C.˜120° C. for 4 hours. The reaction medium is allowed to return to ambient temperature, filtered, rinsed and evaporated to dryness. A product is obtained which is chromatographed on silica eluting with an ethyl acetate-cyclohexane mixture 1-1. In this way, 1.19 g of sought product is obtained.

Stage E: methyl 3-[4-[2,4-dihydro-2-(1-methylpropyl)-3-oxo-3H-1,2,4-triazol-4-yl]phenyl]-2(E)-propenoate.

A mixture of 1.14 g of methyl 3-(tributylstannyl)-2(E)-propenoate, 1.15 g of the product of the previous stage and 64 mg of dichlorobis (triphenylphosphine)-palladium (PdCl₂(PPh₃)₂) is taken to 110° C. for 30 minutes. 4 ml of a saturated solution of sodium acid carbonate, 0.31 g of potassium fluoride, 4 ml of dimethylformamide (DMF) are added followed by agitating for 1 hour at ambient temperature, filtering, rinsing, decanting and extracting with methylene chloride. After drying, filtering and evaporating to dryness, 1.7 g of sought product is obtained.

Stage F: 2,4-dihydro-4-[4-(3-hydroxy-l(E)-propenyl)phenyl]-2-(1-methylpropyl)-3H-1,2,4-triazol-3-one.

4.4 ml of 1.5M diisobutylaluminium hydride (DIBAH) in toluene is added to a solution of 0.67 g of the product of the previous stage in 7 ml of THF. Agitation is carried out at ambient temperature for 1 hour. The reaction medium is taken to 0° C. and an aqueous solution of THF (1-1) is poured in, followed by filtering, rinsing, decanting, extracting with methylene chloride, drying, filtering and evaporating to dryness. 0.547 g of product is obtained which is chromatographed on silica eluting with an ethyl acetate-cyclohexane mixture 1-1. 0.275 g of sought product is obtained.

Stage G: 3-[4-[2,4-dihydro-2-(1-methylpropyl)-3-oxo-3H-1,2,4-triazol-4-yl]phenyl]-2(E)-propenal.

A mixture of 0.268 g of the product of the previous stage, 5 ml of methylene chloride and 1.10 g of manganese oxide is agitated at ambient temperature for 3 hours. After filtering, drying and evaporating to dryness, 0.244 g of product is obtained which is used as it is.

Preparation 2: Cis (±)6-[[2-(2,4 dichlorophenyl)-2-(1H-imidazol-1-yl-methyl)-1,3-dioxolan-4-yl]-methoxy]-1,2,3,4-tetrahydro-isoquinoline

Stage A:

A suspension containing 5.8 g of 1,2,3,4 tetrahydroisoquinoline 6-OH and 50 ml of THF is agitated for 15 hours at ambient temperature. 11.13 g of diterbutyl dicarbonate in 25 ml of THF is added at 20° C. The reaction medium is poured into an ice-cold solution of potassium acid carbonate followed by extracting with ethyl acetate, drying, filtering and concentrating. The product obtained is taken up in pentane, crystallization is initiated and the crystals obtained are washed with pentane. 9.26 g of sought product is obtained. M.p.=114° C.

Stage B:

5.4 g of sodium hydride at 55≅60% in dispersion in oil is introduced over 20 minutes at ambient temperature into a solution of 24.63 g of the product prepared in Stage A and 250 ml of DMF. The reaction medium is taken to 55° C. for 2 hours, then allowed to return to ambient temperature and 54.6 g of cis-(+)-2-(2,4-dichlorophenyl)-2-(1H-imidazol-1-yl-methyl)-1,3-dioxolane-4-methanol methane sulphonate is introduced. The reaction mixture is taken to 80° C. for 20 hours. Agitation is then carried out at ambient temperature for 72 hours, followed by pouring onto ice, agitating for 1 hour, decanting, drying, filtering and concentrating. 95.8 g of product is obtained which is chromatographed on silica eluting with a heptane acetone mixture (6/4). In this way, 45 g of sought product is obtained which is used as it is in the following stage.

Stage C: Cis (±)6-[[2-(2,4 dichlorophenyl)-2-(1H-imidazol-1-yl-methyl)-1,3-dioxolan-4-yl]-methoxy]-1,2,3,4-tetrahydroisoquinoline.

45 g of the product prepared in the previous stage in solution in 200 cm³ of ethyl acetate is cooled down to 10° C. 100 cm³ of a solution containing 50 g of ice and 50 cm³ of a 12N hydrochloric acid solution is added. The reaction mixture is agitated for 4 hours then concentrated under reduced pressure, followed by taking up with ethyl acetate. A product is obtained which is taken up in ethyl ether. The product obtained is triturated, separated, rinsed and dried. 60.8 g of product is obtained which is poured into 250 cm³ of water. After cooling down, 50 cm³ of 28% ammonium hydroxide solution is poured in. Agitation is carried out for 30 minutes and 150 cm³ of methylene chloride is added. The reaction mixture is maintained under agitation for 30 minutes, followed by extracting with methylene chloride, washing, drying, filtering and concentrating. 35.68 g of sought product is obtained.

Preparation 3: 3-[4-[2,4-dihydro-2-((1S,2R)-2-hydroxy-1-methylpropyl)-3-oxo-3H-1,2,4-triazol-4-yl]phenyl]-2(E)-propenal.

Stage A: (4R,5R)-4,5-dimethyl-1,3,2-dioxathiolane 1,1-dioxide

A mixture of 0.45 g of 2,3-butanediol, 5 ml of carbon tetrachloride and 0.44 ml of thionyl chloride (SOCl₂) is taken to reflux for 30 minutes, followed by bringing to 0° C. and adding 5 ml of acetonitrile, 1.5 mg of RuCl₃ and 1.6 g of NaIO₄. The reaction medium is maintained under agitation at ambient temperature for 2 hours. After extracting with ethyl ether, washing with water, with a solution of sodium carbonate acid and with sodium chloride, drying, filtering and evaporating to dryness, 0.6 g of product is obtained which is taken up in a mixture of 10 ml of CH₃CN, 7 mg of ruthenium chloride (RuCl₃), 1.6 g of sodium periodate (NaIO₄) and 1 ml of water. Agitation is carried out for 2 hours at ambient temperature followed by extracting with methylene chloride, washing with water, drying, filtering and evaporating to dryness. After taking up in methylene chloride, filtering and evaporating to dryness, 0.539 g of sought product is obtained.

Stage B: 2,4-dihydro-2-((1S,2R)-2-hydroxy-1-methylpropyl)-4-(4-iodophenyl)-3H-1,2,4-triazol-3-one.

A mixture of 0.861 g of 2,4-dihydro-4-(4-iodophenyl)-3H-1,2,4-triazol-3-one (Stage C of Preparation 1), 8 ml of methyl-isobutylketone, 0.829 g of K2CO3, 86 mg of Aliquat 336 and 0.913 g of the product of the previous stage is taken to 110° C. for 2 hours 30 minutes. The reaction medium is allowed to return to ambient temperature and water is added followed by decanting, washing with water, evaporating to dryness and taking up in 6 ml of a hydrobromic acid solution (32%). The reaction medium is heated at 50° C. for 2 hours then returned to ambient temperature, followed by extracting with methylene chloride, drying, filtering and evaporating to dryness. 0.316 g of sought product is obtained.

Stage C: methyl 3-[4-[2,4-dihydro-2-((1S,2R)-2-hydroxy-1-methylpropyl)-3-oxo-3H-1,2,4-triazol-4-yl]phenyl]-2(E)-propenoate.

By operating as previously (Stage E Preparation 1), the sought product was obtained.

Stage D: 2,4-dihydro-2-((1S,2R)-2-hydroxy-1-methylpropyl)-4-[4-(3-hydroxy-1(E)-propenyl)phenyl]-3H-1,2,4-triazol-3-one.

By operating as previously (Stage F Preparation 1), the sought product was obtained.

Stage E: 3-[4-[2,4-dihydro-2-((1S,2R)-2-hydroxy-1-methyl-propyl)-3-oxo-3H-1,2,4-triazol-4-yl]phenyl]-2(E)-propenal.

By operating as previously (Stage G Preparation 1), the sought product was obtained.

Preparation 4: (E)-2-[3-(4-chlorophenyl)-2-propenyl]-1,2,3,4-tetrahydro-6-isoquinolinol.

A mixture of 2 g of 1,2,3,4-tetrahydro-6-isoquinolinol and 2.5 g of (E)-1-chloro-4-[3-chloro-1-propenyl]-benzene, 2 g of potassium carbonate and 50 ml of DMF is agitated for 36 hours at 25° C. The DMF is driven off under reduced pressure. The residue is taken up in a mixture of methylene chloride and water. After filtering, 2.5 g of sought product is obtained. M.p.=233-234° C.

Preparation 5: trans (±)-2-(4-chlorophenyl)-cyclopropane carboxaldehyde.

8.1 ml of triethylamine is added to a solution of 1.4 g of trans (±)-2-(4-chlorophenyl)-cyclopropane ethanol and 7 ml of DMSO. 3.2 g of an S₀₃ pyridine solution is added at a temperature lower than or equal to 25° C. Agitation is maintained and the reaction medium is poured into a water-ice mixture, followed by extracting with ether, drying and concentrating. After chromatography on silica eluting with a hexane-ethyl acetate mixture 6-2, 76 mg of sought product is obtained.

Preparation 6: Cis (±)6-[[2-(2,4 dichlorophenyl)-2-(1H-imidazol-1-ylmethyl)-1,3-dioxolan-4-yl]-methoxy]-1,2,3,4-tetrahydro-2-[3-tributylstannyl)-2(E)-propenyl]-isoquinoline

Stage A: 3-(tributylstannyl)-2(E)-propen-1-ol

12 g of methyl 3-(tributylstannyl)-2(E)-propenoate is dissolved in 100 ml of THF. The reaction medium is cooled down to −78° C. and 67 ml of dibutylaluminium hydride is added. The reaction medium is taken to a temperature of 0° C., poured into methanol, water is added and agitation is carried out overnight. The aluminium salts are filtered followed by washing with ethyl acetate, decanting the organic phases, drying and concentrating. 9 g of product is obtained which is purified by chromatography eluting with a hexane ethyl acetate mixture 8/2. 7.25 g of the sought product is obtained.

Stage B: (3-bromo-1-(E)-propenyl)tributyl-stannane

A solution of 5.5 g of triphenylphosphine in 10 ml of methylene chloride is introduced dropwise over 30 minutes at 0° C. into a solution containing 5.2 g of the product obtained in Stage A in 50 ml of methylene chloride and 6 g of carbon tetrabromide. The reaction medium is maintained at 0° C. for 1 hour then poured into water followed by extracting with methylene chloride, drying, chromatographing on silica eluting with heptane and 5.06 g of sought product is obtained.

Stage C: Cis (±)6-[[2-(2.4 dichlorophenyl)-2-(1H-imidazol-1-yl-methyl)-1,3-dioxolan-4-yl]-methoxy]-1,2,3,4-tetrahydro-2-[3-tributylstannyl)-2(E)-propenyl]-isoquinoline.

A solution of 5.06 g of the product prepared previously and 20 ml of acetone is introduced dropwise under agitation and a nitrogen atmosphere into a mixture comprising 5.68 g of Cis (±)6-[[2-(2,4-dichlorophenyl)-2-(1H-imidazol-1-yl-methyl)-1,3-dioxolan-4-methyl]-methoxy]-1,2,3,4-tetrahydroisoquinoline prepared according to Patent No. WO 0020413, 1.5 g of Ag₂O, 100 ml of acetone and 50 ml of DMF. The reaction medium is maintained at ambient temperature overnight followed by filtering, taking up in water, extracting with ethyl acetate and purifying by chromatography on silica (eluent: methylene chloride, methanol (95-5). 6.66 g of sought product is obtained.

### EXAMPLE 1

### 2,4-dihydro-4-[4-[3-[6-[[(cis)-2-(2,4-dichlorophenyl)-2-(1H-imidazol-1-ylmethyl)-1,3-dioxolan-4-yl]-methoxy]-1,2,3,4-tetrahydro-2-isoquinolinyl]-1(E)-propenyl]-phenyl]-2-(1-methylpropyl)-3H-1,2,4-triazol-3-one.

A mixture of 0.325 g of the product of Preparation 2, 5 ml of methanol, 0.23 g of the product of Preparation 1 and 121 μl of acetic acid is agitated for 1 hour at ambient temperature. 0.067 g of NaBH₃CN is added. Agitation is carried out for 3 hours at ambient temperature followed by taking up in a mixture of methylene chloride and water. 0.4 ml of ammonium hydroxide is added. After decanting, extracting with methylene chloride, drying, filtering and evaporating to dryness, 0.594 g of product is obtained which is chromatographed on silica eluting with a methylene chloride-isopropanol-ammonium hydroxide mixture (94-6-0.3). 0.194 g of sought product is obtained.

MS: M⁺H⁺=715, 460, 256, 200.

### EXAMPLE 2

### 4-[4-[3-[6-[[(cis)-2-(2,4-dichlorophenyl)-2-(1H-imidazol-1-ylmethyl)-1,3-dioxolan-4-yl]methoxy]-1,2,3,4-tetrahydro-2-isoquinolinyl]-1(E)-propenyl]phenyl]-2-((1S,2R)-2-hydroxy-1-methylpropyl)-3H-1,2,4-triazol-3-one

By operating as in Example 1, starting from the product of Preparation 3, the sought product was obtained.

### EXAMPLE 3

### cis-2-[3-(4-chlorophenyl)-2(E)-propenyl]-6-[[2-(2,4-dichlorophenyl)-2-(1H-imidazol-1-yl-methyl)-1,3-dioxolan-4-yl]-methoxy]-1,2,3,4-tetrahydro-5-isoquinolin-amine.

Stage A:

3 g of (E)-2-[3-(4-chlorophenyl)-2-propenyl]-1,2,3,4-tetrahydro-6-isoquinolinol is suspended in 20 ml of ethanol. 4.12 g of hydrated ferric nitrate Fe(NO₃)_{3.}9H₂O is added followed by heating at 40° C. overnight. The reaction is hydrolyzed with water and 1N HCl. The precipitate is filtered and the resulting aqueous phases are extracted with CH₂Cl₂. The precipitate and the dry extract are combined and the mixture is chromatographed on silica eluting with a CH₂Cl₂-iPrOH mixture (95-5) in order to separate the 5 and 7 nitro isomers. 2.9 g of product is obtained.

Stage B: cis-2-[3-(4-chlorophenyl)-2(E)-propenyl]-6-[[2-(2,4-dichlorophenyl)-2-(1H-imidazol-1-yl-methyl)-1,3-dioxolan-4-yl]-methoxy]-5-nitro-1,2,3,4-tetrahydro-isoquinoline

240 mg of the product of Stage A and 372.5 mg of cis-(±)-2-(2,4-dichlorophenyl)-2-(1H-imidazol-1-yl-methyl)-1,3-dioxolan-4-methanol toluenesulphonate, 24 ml of methylisobutylketone, 290 mg of potassium carbonate, 74.5 mg of Aliquat 336 and 240 ml of water are heated at 80° C. for 8 hours. The reaction medium is left to cool down, filtered and rinsed. The filtrate is concentrated under reduced pressure and an oil is obtained which is purified on silica, eluting with a methylene chloride-isopropanol mixture (94-6). 400 mg of sought product is obtained.

Stage C: cis-2-[3-(4-chlorophenyl)-2(E)-propenyl]-6-[[2-(2,4-dichlorophenyl)-2-(1H-imidazol-1-yl-methyl)-1,3-dioxolan-4-yl]-methoxy]-1,2,3,4-tetrahydro-5-isoquinolin-amine.

A mixture of 4.51 g of the product of the previous stage, 30 ml of ethanol, 33 g of FeSO_{4.}7H₂O, 210 ml of water and 130 ml of concentrated ammonium hydroxide at 20% is heated to 80°˜85° C. for 8 hours. After filtering and rinsing with ethyl acetate, the solid is triturated in ethyl acetate and in potassium acid carbonate and filtered. After decanting and drying, 4.2 g of crude sought product is obtained which is chromatographed on silica eluting with a CH₂Cl₂-iPrOH—NH₃ mixture (92-0.8-0.3) and 2.42 g of sought product is obtained. MS: M⁺H⁺=625.

### EXAMPLE 4

### cis-7-chloro-2-[3-(4-chlorophenyl)-2(E)-propenyl]-6-[[2-(2,4-dichlorophenyl)-2(1H-imidazol-1-yl-methyl)-1,3-dioxan-4-yl]-methoxy]-1,2,3,4-tetrahydro-isoquinoline.

328 mg of the compound of the previous example (Stage B) is introduced into a mixture of 3 ml of a 20% aqueous solution of TiCl₃ and 5 ml of methanol. Agitation is carried out for 15 hours at ambient temperature followed by pouring into sodium acid carbonate, evaporating the methanol and extracting with methylene chloride. Chromatography is carried out on silica eluting with toluene-isopropanol (92-8) with 0.1% ammonium hydroxide. 117 mg of sought product is isolated. MS: M⁺H⁺=659 (4 Cl).

### EXAMPLE 5

### cis-1-[2-[3-(4-chlorophenyl)-2(E)-propenyl]-6-[[2-(2,4-dichlorophenyl)-2(1H-imidazol-1-yl-methyl)-1,3-dioxolan-4-yl]-methoxy]-1,2,3,4-tetrahydro-isoquinolinyl]-2-pyrrolidinone.

A mixture of 125 mg of the product of Example 3, 1 ml of methylene chloride, 40 mg of ClCO(CH₂)₃Br and 30 mg of DMAP in 1 ml of methylene chloride is agitated at ambient temperature for 24 hours, followed by pouring into a mixture of potassium acid carbonate and ethyl acetate. 160 mg of crude product is obtained; after chromatography, 80 mg of sought product is obtained. MS: M⁺H⁺=694.

### EXAMPLE 6

### cis-2-[3-(4-chlorophenyl)-2(E)-propenyl]-6-[[2-(2,4-dichlorophenyl)-2(1H-imidazol-1-yl-methyl)-1,3-dioxolan-4-yl]-methoxy]-1,2,3,4-tetrahydro-7-isoquinolinamine.

Stage A: cis-2-[3-(4-chlorophenyl)-2(E)-propenyl]-6-[[2-(2,4-dichlorophenyl)-2(1H-imidazol-1-yl-methyl)-1,3-dioxolan-4-yl]-methoxy]-7-nitro-1,2,3,4-tetrahydro-isoquinoline.

By operating as in Example 3, the sought product is obtained. Rf=0.35 (CH₂Cl₂-isopropanol 95-5).

Stage B: cis-2-[3-(4-chlorophenyl)-2(E)-propenyl]-6-[[2-(2,4-dichlorophenyl)-2(1H-imidazol-1-yl-methyl)-1,3-dioxolan-4-yl]-methoxy]-1,2,3,4-tetrahydro-7-isoquinolinamine.

By operating as in Example 3 starting from the product of the previous stage, the sought product was obtained.

### EXAMPLE 7

### cis-2-[3-(4-chlorophenyl)-2(E)-propenyl]-6,7-bis-[[2-(2,4-dichlorophenyl)-2(1H-imidazol-1-yl-methyl)-1,3-dioxolan-4-yl]-methoxy]-1,2,3,4-tetrahydro-isoquinoline.

By operating as previously starting from 1,2,3,4-tetrahydroisoquinoline 6,7-diol, (E)-2-[3-(4-chlorophenyl)-2-propenyl]-1,2,3,4-tetrahydro-5,6-iso-quinolin-diol is obtained which is condensed with cis-2-(2,4-dichlorophenyl)-2-(1H-imidazol-1-yl-methyl)-1,3-dioxolan-4-methanol toluenesulphonate in order to obtain the sought product. Rf=0.37 (CH₂Cl₂-methanol 9-1).

### EXAMPLE 8

### cis-2-[2-trans-(4-chlorophenyl)-cyclopropyl]-methyl]-6-[[2-(2,4-dichlorophenyl)-2(1H-imidazol-1-yl-methyl)-1,3-dioxolan-4-yl]-methoxy]-1,2,3,4-tetrahydro-isoquinoline.

A mixture of 76.5 mg of the product of Preparation 5, 175 mg of the product of Preparation 2, 76 ml of acetic acid, 3 ml of methanol is agitated for 15 minutes at ambient temperature. 27 mg of sodium cyanoborohydride (NaBH₃CN) is added. Agitation is carried out for 15 hours. The pH is adjusted to 8.9 with ammonium hydroxide followed by extracting with methylene chloride, washing with water, drying and concentrating. A product is obtained which is chromatographed on silica eluting with a hexane-ethyl acetate mixture (8-2). The reaction medium is concentrated and 123 mg of sought product is obtained.

MS: M⁺H⁺=624, 460, 165.

### EXAMPLE 9

### cis-2-[3-[4-[(2-methoxyethoxy)methoxy]phenyl]-2( [symbol] , propenyl]-6-[[2-(2,4-dichlorophenyl)-2(1H-imidazol-1-yl-methyl)-1,3-dioxolan-4-yl]-methoxy]-1,2,3,4-tetrahydro-isoquinoline.

A solution comprising 2.3 g of the product obtained as in Preparation 2 and 1.3 g of 3-[4-[(2-methoxyethoxy) methoxy]phenyl]-2(E)-propenal in 40 ml of methanol is adjusted to pH 6 by adding 250 ml of acetic acid. 930 mg of sodium cyanoborohydride is then added and agitation is carried out for 16 hours at ambient temperature. After concentrating under reduced pressure, taking up in sodium acetate, washing with an aqueous solution of sodium hydroxide then with a saturated aqueous solution of sodium chloride, drying and concentrating to dryness under reduced pressure, 4.1 g of product is collected which is chromatographed on silica (eluent CH₂Cl₂/MeOH 93-7) and 2.52 g of expected product is obtained.

rf=0.30 (CH₂Cl₂/MeOH 93-7).

### EXAMPLE 10

### cis-4-[3-[6-[[2-(2,4-dichlorophenyl)-2-(1H-imidazol-1-yl-methyl)-1,3-dioxan-4-yl]-methoxy]-1,2,3,4-tetrahydro-2-isoquinolinyl]-1(E)-propenyl]-phenol.

2 g of the product obtained in Example 9 in 20 ml of methylene chloride is cooled down to 0° C. and 20 ml of trifluoroacetic acid is added. Agitation is carried out for 30 minutes at 0° C. then for 30 minutes at ambient temperature, followed by concentrating to dryness under reduced pressure, taking up the residue in methylene chloride, adding 20 ml of water and cooling down to 0° C. Concentrated ammonium hydroxide is added to pH=10 followed by extracting with methylene chloride, washing with water, drying and eliminating the solvents under reduced pressure. 1.81 g of expected product is collected which is used as it is for the following stage.

rf=0.40 (CH₂Cl₂/Methanol 90-10).

NMR (CDCl₃): 2.81 (m) 2H: CH₂ in position 10; 2.89 (m) 2H: CH₂ in position 9; 3.25 (dd) 3.68 (dd) 2H-3.74 (dd) 3.86 (dd) 2H: OCH₂—CH—CH₂—O; 4.36 (m) 1H: OCH₂—CH—CH₂—O; 4.41 4.52 AB 2H: N—CH₂—Cq; 3.31 (bd) 2H: N—CH₂—CH═CH-Φ; 6.10 (td) 1H: N—CH₂—CH═CH-Φ J=6.5 16 HZ; 6.48 (bd) 1H: N—CH₂—CH═CH-Φ J=16 HZ; 3.65 (bs) 2H: CH₂ in position 2; 6.54 (d) 1H: H7; 6.60 (dd) 1H: H5; 6.90 (d) 1H: H4; 6.79-7.12 AA′BB′ O-Φ; 7.47 (d) 1H: Ha; 7.27 (masked): Hb; 7.61 (d) 1H: Hc; 7.58 (s) 1H: H2′; 7.00 (d) 1H: H4′ and H5′.

### EXAMPLE 11

### mono[cis-4-[3-[6-[[2-(2,4-dichloro phenyl)-2-(1H-imidazol-1-yl-methyl)-1,3-dioxan-4-yl]-methoxy]-1,2,3,4-tetrahydro-2-isoquinolinyl]-1(E)-propenyl]-phenyl] phosphate and bis(phenylmethyl) phosphate.

A solution comprising 592 mg of the product obtained in Example 10 in 20 ml of methylene chloride is cooled down to 0° C. then 960 μl of carbon tetrachloride, 24 mg of dimethylaminopyridine and 732 μl of diisopropyl ethylamine then dropwise 628 μl of dibenzylphosphite are added. Agitation is maintained for 3 hours at 0° C. and 20 ml of a molar solution of sodium hydrogen phosphate is added, the reaction medium is left to return to ambient temperature, extraction is carried out with methylene chloride followed by washing with an aqueous solution of sodium chloride and the solvents are eliminated under reduced pressure. 725 mg of crude product is obtained which is purified by chromatography on silica (eluent CH₂Cl₂/MeOH 93-7) and 360 mg of pure expected product is collected.

rf=0.35 (CH₂Cl₂/MeOH 93-7).

### EXAMPLE 12

### [cis-4-[3-[6-[[2-(2,4-dichloro phenyl)-2-(1H-imidazol-1-yl-methyl)-1,3-dioxan-4-yl]-methoxy]-1,2,3,4-tetrahydro-2-isoquinolinyl]-1(E)-propenyl]-phenyl] phosphate (trifluoroacetate salt).

360 mg of the product obtained in Example 11 in 5 ml of methylene chloride and 5 ml of trifluroacetic acid is agitated for 5 hours at ambient temperature. The solvents are eliminated under reduced pressure and 352 mg of crude product is recovered which is purified by chromatography on silica (eluent CH₃CN/CH₂Cl₂/TFA 40-60-0.03) and after lyophilization 118 mg of the expected product and 26 mg of the monobenzylated intermediate phosphate of mono[cis-4-[3-[6-[[2-(2,4-dichloro phenyl)-2-(1H-imidazol-1-yl-methyl)-1,3-dioxan-4-yl]-methoxy]-1,2,3,4-tetrahydro-2-isoquinolinyl]-1(E)-propenyl]-phenyl] and mono(phenylmethyl) is obtained (trifluoroacetate salt)

expected product

rf=3.70 (CH₃CN/H₂O-TFA 40-60-0.03)

monobenzylated product

rf=5.08 (CH₃CN/H₂O 40-60)

NMR (DMSO) of the expected product. 3.10 (t) 3.46 (masked): N—CH₂—CH₂—CH—CH—CH₂; 4.66 (s): N—CH₂—CH₂—CH—CH—CH₂: 3.96 (m) CH₂—CH═CH—: ΔE; 6.30 (dt): CH₂—CH═CH—; 6.85 (d,J=16): CH₂—CH═CH—; 3.72 (m) 3.94 (m): O—CH₂—CH—CH₂—O; 4.38 (m): O—CH₂—CH—CH₂—O; 3.75 (m) 3.84 (m): O—CH₂—CH—CH₂—O; 7.45 (dd) 7.61 (d) 7.64 (d): dichlorophenyl; 7.12 (bs) 7.23 (bs) 8.10 (bs): CH imidazole; 4.29 (s): N—CH₂; 6.79 (dd) 6.82 (d) 7.14 (d) and 7.20 7.46: CH of the phenyl nuclei.

### EXAMPLE 13

### mono[cis-4-[3-[6-[[2-(2,4-dichloro phenyl)-2-(1H-imidazol-1-yl-methyl)-1,3-dioxan-4-yl]-methoxy]-1,2,3,4-tetrahydro-2-isoquinolinyl]-1(E)-propenyl]-phenyl] sulphonate (N,N-diethylethanamine Salt).

236 mg of the product obtained as in Example 10 in 4 ml of dimethylformamide is agitated for 16 hours at ambient temperature in the presence of 1 ml of pyridine and 1.5 g of N,N-dimethylformamide trioxide sulphide complex. 20 ml of ether is then added, the supernatant phase is eliminated and a further 20 ml of ether is added. Agitation is carried out for a few minutes, the precipitate is separated and 680 mg of crude product is recovered which is purified by carrying out chromatography twice on silica (eluent CH₂Cl₂/MeOH/TEA 87-13-1). 239 mg of expected product is obtained

rf=0.40 (CH₂Cl₂/MeOH/TEA 87-13-1).

NMR: (CDCl₃) 1.27 (t): N—(CH₂CH₃)₃; 2.98 (q): N—(CH₂CH₃)₃; 3.31 (bd) 2H: N—CH₂CH═CH-Φ; 6.26 (td) 1H: N—CH₂—CH═CH-Φ J=166.5 HZ; 6.56 (dd) 1H: N—CH₂—CH═CH-Φ J=16 HZ ΔE; 3.32 (m) 3.72 (dd) 2H-3.75 (dd) 3.88 (dd) 2H: O—CH₂—CH—CH₂—O; 4.35 (m) 1H: O—CH₂—CH—CH₂—O; 4.40 4.51 AB 2H: N—CH₂—Cq; 3.60 (bs) 2H: CH₂ in position 2; 7.25 (dd) 1H: Hb; 7.46 (d) 1H: Ha; 7.50 (bs) 1H: H_{2′; 7.57} (d) 1H: Hc; 6.61 (dd) 1H: H₅; 6.57 (bs) 1H: H₇; 6.97 (bd) 2H: H₄′ and H_{5′; 6.92} (d) 1H: H₄; 7.34 (m) 4H: Φ 2.77 (bt) 2H: CH₂ in position 9; 2.91 (bt) 2H: CH₂ in position 10.

By operating as previously, the following products were prepared:

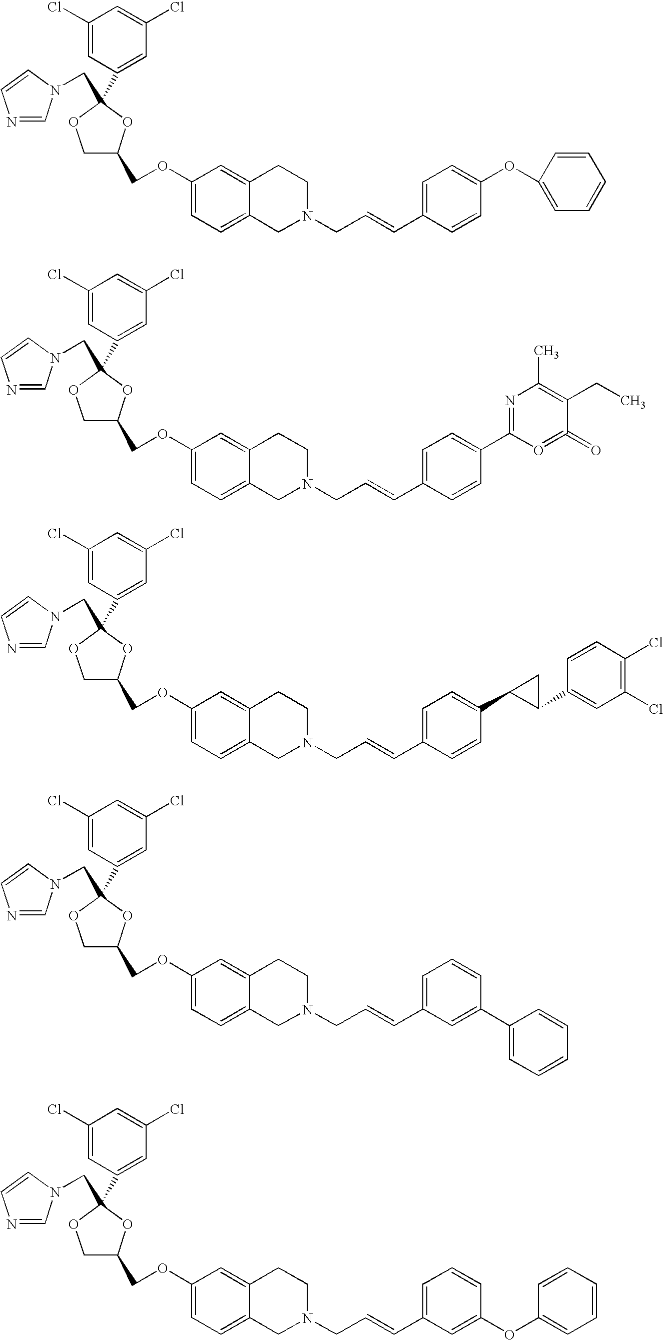

By operating as previously, the following products were prepared:

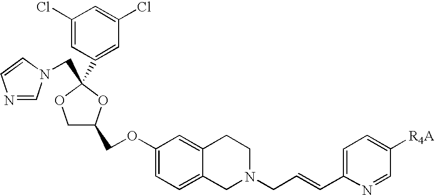

R4A for example taking the value

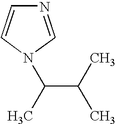

Products were also prepared corresponding to the formula

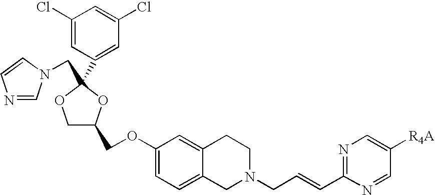

Pharmaceutical Compositions

Compounds were prepared containing

| | |
|---|---|
| Product of Example 1 | 50 mg |
| Excipient s.q.f. | 1 g |

Detail of the excipient: starch, talc, magnesium stearate.

Biological Activity

Antifungal activity of the product of Example 1 or product P.

Female mice were used weighing 18 to 22 g. A quantity of Candida Albicans 44858 at a rate of 106 CFU per mouse (CFU: colony forming unit) is administered into the vein of the tail. The mice are separated into 5 batches of 5 mice and they are treated as follows:

1 hour after infection

group 1: the mice are treated with product P 25 mg/kg by oral route

group 2: the mice are treated with product P by intraperitoneal route at a rate of 25 mg/kg

group 3: the mice are treated with fluconazole (25 mg/kg by oral route).

group 4: the mice are treated with fluconazole (25 mg/kg by intraperitoneal route).

group 5: the mice receive no antifungal treatment.

For a period of 22 days, the dead mice are counted.

Conclusion

The product at the dose used in the 2 administration methods used present an excellent activity.

Moreover the test was carried out with an administration under the order as those obtained with fluconazole.

The same treatments are also carried out in the “topical model” with dermal fungus for example trichophyton and in the sublethal model.

Minimum Inhibitory Concentration (MIC)

Candida albicans cells are prepared as indicated in the Journal of Antimicrobial Chemotherapy 38, 579-587, washed 3 times with a 0.1 M solution of phosphate and used immediately in order to determine the minimum inhibitory concentration (MIC).

The MIC's are determined by the modification of a microtitre plate according to the standard method of the Comité national des standards cliniques de laboratoire [National Committee for clinical laboratory standards].

RPMI-1640 and L-glutamine buffered to pH 7 with a 0.15 M solution of MOPS (3-[N-morpholino]propane sulphonic acid) are used as medium. Candida albicans cells (1.5×10³ cells/ml) are added to the wells of a 96-well plate containing RPMI-1640 and the dilutions of antifungal agents. The results are read 48 hours after incubation at 35° C. and the MIC or minimum inhibitory concentration which inhibits the growth of the Candida albicans cells is determined.

Minimum Fungicidal Concentration

After reading the MIC at 48 hours, the plates are shaken and 10 μL of aliquot is removed from the wells, which is placed on rectangular disks containing agar dextrose. The plates are incubated for 48 hours at 35° C.; the minimum fungicidal concentration and the concentration of antifungal agent at which the number of units forming colonies is zero.

1) In all possible stereoisomeric forms, as well as their mixtures, the compounds of formula (I): 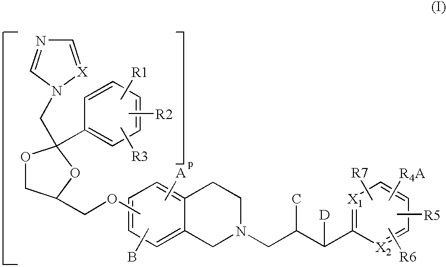
in which
p represents the number 1 or 2,
X, X₁ and X₂, identical or different, represent a nitrogen atom or a CH═ radical,
R₁, R₂, R₃, R₅ and R₆, identical to or different from each other, in any position on the rings which carry them, represent a hydrogen atom, a halogen atom, an alkyl, O-alkyl, S(O)ₙ-alkyl, alkenyl, O-alkenyl, S(O)ₙ-alkenyl, alkynyl, O-alkynyl, S(O)ₙ-alkynyl radical containing up to 8 carbon atoms, optionally substituted by one or more halogen atoms, n representing the number 0, 1 or 2, or represent an NO₂, NH₂ or C≡N radical, R₁, R₂ and R₃ on the one hand and R₅ and R₆ on the other hand being able to form rings two by two,
R₇ represents a hydrogen atom or an OH, OSO₃H or OPO(OH)₂ radical,
R₄A represents an R₄ radical which can take one of the values indicated above for R₁, R₂, R₃, R₅ or R₆, and also being able to represent a non substituted or substituted heterocycle, a non substituted or substituted aryl or O-aryl group containing up to 14 carbon atoms, a cycloalkyl containing 3 to 6 carbon atoms, non substituted or substituted by an aryl optionally substituted by one or more halogen atoms and also being able to represent an oxygenated or nitrogenous chain connected to the phenyl or heteroaryl nucleus by an oxygen or nitrogen atom,
A and B, identical to or different from each other, represent a hydrogen atom or an oxygenated or nitrogenous chain connected to the phenyl nucleus by an oxygen or nitrogen atom,
C and D, identical to or different from each other, represent a hydrogen or halogen atom or an alkyl radical containing up to 8 carbon atoms, optionally substituted by one or more halogen atoms or, together with the carbons which carry them, form a ring optionally substituted by one or more halogen atoms, one or more alkyl radicals containing up to 8 carbon atoms or together form a double bond, with the exception of the compounds corresponding to formula (IA): 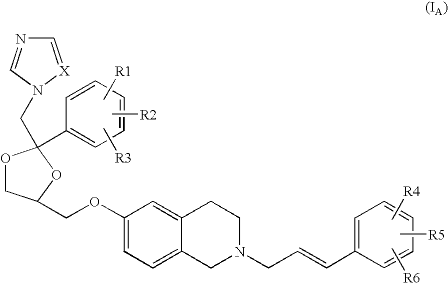
in which the various substituents retain their previous meaning, as well as their addition salts with acids.

2) The compounds of general formula (I) defined in claim 1, corresponding to the formula: 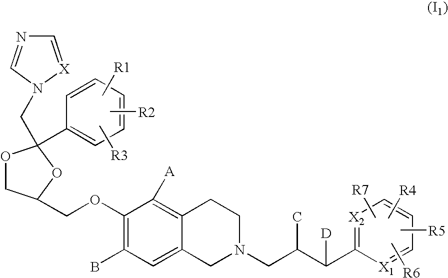
in which X, X₁ and X₂ identical or different represent a nitrogen atom or a —CH═ radical, R₁, R₂, R₃, R₅, R₆, R₇ identical or different being in any position on the rings which carry them.

3) The compounds of formula (I) defined in claim 1 or 2, in which X, X₁ and X₂ represent a —CH═ radical.

4) The compounds of formula (I) defined in claim 1, 2 or 3, in which X represents a CH═ radical and either X₁, or X₂ represents a nitrogen atom.

5) The compounds of formula (I) defined in any one of claims 1 to 4, in which R₁ and R₂ represent a halogen atom.

6) The compounds of formula (I) defined in one of claims 1 to 4, in which R₄ is a halogen atom.

7) The compounds of formula (I) defined in claim 5 or 6, in which the halogen is a chlorine atom.

8) The compounds of formula (I) defined in any one of claims 1 to 5, in which R₃ represents a hydrogen atom.

9) The compounds of formula (I) defined in any one of claims 1 to 8, in which C and D form a double bond.

10) The compounds of formula (I) defined in claim 9, in which the geometry of the double bond is E.

11) The compounds of formula (I) defined in any one of claims 1 to 10, in which the dioxoxaryl radical is in cis position.

12) The compounds of formula (I) defined in any one of claims 1 to 8 in which R₇ represents an OH, OSO₃H or OPO(OH)₂ radical.

13) The compounds of formula (I) defined in any one of claims 1 to 12, in which A or B does not represent a hydrogen atom.

14) The compounds of formula (I) defined in claim 13, in which A or B represents a
W(CO)ᵣ(CH₂)ₛZ
radical in which r represents the number 0 or 1, s represents an integer varying from 0 to 6, W represents an oxygen atom or an —N(R₁₁)— radical, R₁₁ representing a hydrogen atom or an alkyl radical containing up to 8 carbon atoms, R₁₁ also being able to form a ring together with the nitrogen atom which carries it and another atom of the (CO)ᵣ(CH₂)ₛ—Z chain, Z represents a hydrogen atom, an SO₃H or OSO₃H, PO(OH)₂ or OPO(OH)₂, or CO₂H radical as well as the alkaline, alkaline earth salts or salts of amines of these radicals, or a 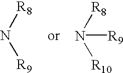
radical
R₈ and R₉ representing a hydrogen atom, an alkyl radical containing up to 8 carbon atoms, R₁₀ representing an alkyl radical containing up to 8 carbon atoms, R₈ and R₉ being able to form a ring optionally containing another heteroatom, and optionally being substituted, or Z represents an optionally substituted heterocyclic radical.

15) The compounds of formula (I) defined in claim 14, in which W represents a nitrogen atom.

16) The compounds of formula (I) defined in claim 1 the names of which follow:
cis-2-[3-(4-chlorophenyl)-2(E)-propenyl]-6-[[2-(2,4-dichlorophenyl)-2-(1H-imidazol-1-yl-methyl)-1,3-dioxolan-4-yl]-methoxy]-1,2,3,4-tetrahydro-5-isoquinolinamine,
cis-2-[3-(4-chlorophenyl)-2(E)-propenyl]-6-[[2-(2,4-dichlorophenyl)-2(1H-imidazol-1-yl-methyl)-1,3-dioxolan-4-yl]-methoxy]-1,2,3,4-tetrahydro-7-isoquinolinamine,
cis-4-[3-[6-[[2-(2,4-dichlorophenyl)-2-(1H-imidazol-1-yl-methyl)-1,3-dioxan-4-yl]-methoxy]-1,2,3,4-tetrahydro-2-isoquinolinyl]-l(E)-propenyl]-phenol,
[cis-4-[3-[6-[[2-(2,4-dichloro phenyl)-2-(1H-imidazol-1-yl-methyl)-1,3-dioxan-4-yl]-methoxy]-1,2,3,4-tetrahydro-2-isoquinolinyl]-1(E)-propenyl]-phenyl]phosphate (trifluoroacetate salt),
mono[cis-4-[3-[6-[[2-(2,4-dichloro phenyl)-2-(1H-imidazol-1-yl-methyl)-1,3-dioxan-4-yl]-methoxy]-1,2,3,4-tetrahydro-2-isoquinolinyl]-1(E)-propenyl]-phenyl]sulphonate (N,N-diethylethanamine salt).

17) As medicaments, the compounds of formula (I) defined in any one of claims 1 to 16, as well as their pharmaceutically acceptable addition salts.

18) The pharmaceutical compositions containing as active ingredient at least one medicament according to claim 17.

19) Process for the preparation of the compounds of formula (I) according to any one of claims 1 to 16, characterized in that a compound of formula (II): 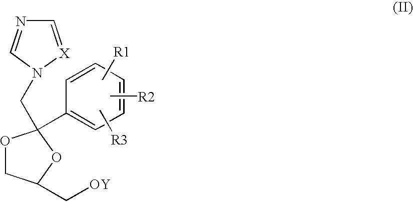
in which:
Y represents a mesyl or tosyl radical and the other substituents retain their previous meaning, is subjected to the action of a compound of formula (III) 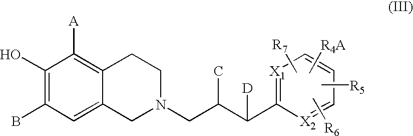
in which the different substituents retain their previous meaning, in order to obtain the corresponding compound of formula (I), which is subjected if desired to the action of a reducing, substitution, addition agent or to the action of an acid in order to obtain the desired compound.

20) As new chemical products, the compounds of formula (III) defined in claim 19.

21) Process for the preparation of the compounds of formula (I) defined in claim 1, characterized in that a compound of formula (IV): 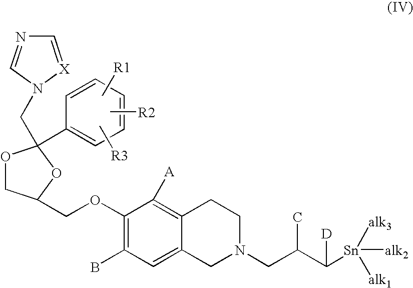
in which alk₁, alk₂ and alk₃ represent an alkyl radical containing up to 8 carbon atoms, R1, R2, R3 and X retain their previous meaning, is subjected to the action of a compound of formula (V): 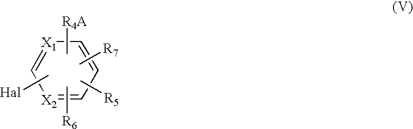
in which Hal represents a halogen atom and the other substituents retain their previous meaning in order to obtain the corresponding compound of formula (I), which is subjected if desired to the action of a reducing, substitution, addition agent or to the action of an acid in order to obtain the desired compound.

22) Process for the compounds of formula (I) defined in claim 1, characterized in that a compound of formula (VI): 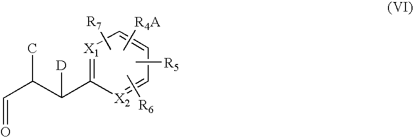
in which the substituents retain the same value as in claim 1, is subjected to the action-of a compound of formula (VII): 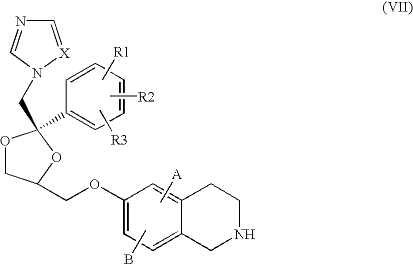
in which the substituents retain the same value as in claim 1, then to the action of a reducing agent in order to obtain the corresponding compound of formula (I) which is subjected if desired to the action of a reducing, substitution, addition agent or to the action of an acid in order to obtain the desired compound.

23) As new chemical products, the compounds of formula (IV) defined in claim 21.

24) As new chemical products the compounds of formula (VI) defined in claim 22.
